(19) United States
(12) Patent Application Publication
(10) Pub. No.: US 2014/0088616 A1
(43) Pub. Date: Mar. 27, 2014

(54) RELEASE MECHANISM FOR HEMOSTATIC CLIP

(71) Applicant: BOSTON SCIENTIFIC SCIMED, INC., Maple Grove, MN (US)

(72) Inventors: Claude CLERC, Marlborough, MA (US); Dane SEDDON, Boston, MA (US)

(73) Assignee: BOSTON SCIENTIFIC SCIMED, INC., Maple Grove, MN (US)

(21) Appl. No.: 14/033,863

(22) Filed: Sep. 23, 2013

Related U.S. Application Data
(60) Provisional application No. 61/704,693, filed on Sep. 24, 2012.

(51) Int. Cl. A61B 17/08

(52) U.S. Cl. CPC A61B 17/083
USPC 606/142

(57) ABSTRACT

A medical device includes a distal portion sized and shaped for insertion into a body lumen and including a capsule having a lumen extending therethrough and a plurality of windows in a proximal end thereof; a bushing including a channel extending therethrough, a distal end thereof including a plurality of arms extending distally therefrom such that a corresponding one of the arms is releasably engagable with a corresponding one of the windows of the capsule, the arms being biased toward a centerline of the bushing in a release configuration; and a bushing support receivable within the channel of the bushing to apply a radially outward pressure to the arms such that the arms lockingly engage the windows of the capsule, the arms formed of a material selected such that movement of the bushing support out of the channel reverts the arms to the release configuration.

### PRIORITY CLAIM

The present application claims the priority to the U.S. Provisional Application Ser. No. 61/704,693, entitled “RELEASE MECHANISM FOR HEMOSTATIC CLIP” filed on Sep. 24, 2012. The specification of the above-identified application is incorporated herewith by reference.

### BACKGROUND

Pathologies of the gastrointestinal (“GI”) system, the biliary tree, the vascular system and other body lumens and hollow organs are often treated through endoscopic procedures, many of which require active and/or prophylactic hemostasis to control bleeding. Hemostasis clips are often deployed via endoscopes to stop internal bleeding by holding together the edges of wounds or incisions to allow natural healing processes to close the wound. Specialized endoscopic clipping devices are used to deploy the clips at desired locations of the body after which the clip delivery device is withdrawn, leaving the clip within the body.

### SUMMARY OF THE INVENTION

The present invention is directed to a system and method for clipping tissue. A medical device according to the invention comprises a distal portion sized and shaped for insertion into a body lumen and including a capsule extending longitudinally from a proximal end to a distal end and including a lumen extending therethrough, the proximal end of the capsule including a plurality of windows extending therethrough. The medical device also comprises a bushing extending from a proximal end to a distal end and including a channel extending therethrough, the distal end including a plurality of arms extending distally therefrom such that a corresponding one of the arms is releasably engagable with a corresponding one of the windows of the capsule, the arms being biased toward a centerline of the bushing in a release configuration and a bushing support receivable within the channel of the bushing to apply a radially outward pressure to the arms such that the arms engage the windows of the capsule in a locked configuration, the arms formed of a material selected to have a yield stress such that movement of the bushing support out of the channel reverts the arms to the release configuration even when the distal portion is inserted through tortuous paths of a body lumen.

### BRIEF DESCRIPTION OF THE DRAWINGS

FIG. 1 shows a partially cross-sectional side view of a device according to an exemplary embodiment of the present invention; and

FIG. 2 shows a side view of a bushing engaging a distal portion of the device according to an exemplary embodiment of the present invention.

### DETAILED DESCRIPTION

The present invention may be further understood with reference to the following description and the appended drawings, wherein like elements are referred to with the same reference numerals. The present invention relates to devices for hemostatic clipping and, in particular, to a hemostatic clip deployed through a single stage process. Exemplary embodiments of the present invention describe a bushing including arms at a distal end thereof to permit releasable attachment of the bushing to a capsule of a hemostasis clipping device as described, for example, in U.S. Patent Application Publication. No. 2008/0306491 to Cohen et al. and entitled “Single Stage Hemostasis Clipping Device” and U.S. Patent Application Publication No. 2011/0046651 to Cohen et al. and entitled “Multifunctional Core for Two-Piece Hemostasis Clip,” the entire disclosures of which are hereby incorporated by reference. The exemplary bushing of the present invention connects a capsule including a hemostatic clip to a flexible delivery member of a clip delivery system, which improves a deployment mechanism for separation of the capsule from the flexible delivery member. The exemplary embodiment of the present invention includes a bushing formed to ensure that, when the clip is deployed, arms of the bushing deform elastically to release the clip. Although the exemplary embodiments specifically describe a clipping device, it will be understood by those of skill in the art that the bushing of the present invention may be utilized with any clipping device or medical device requiring detachment from a catheter or tube.

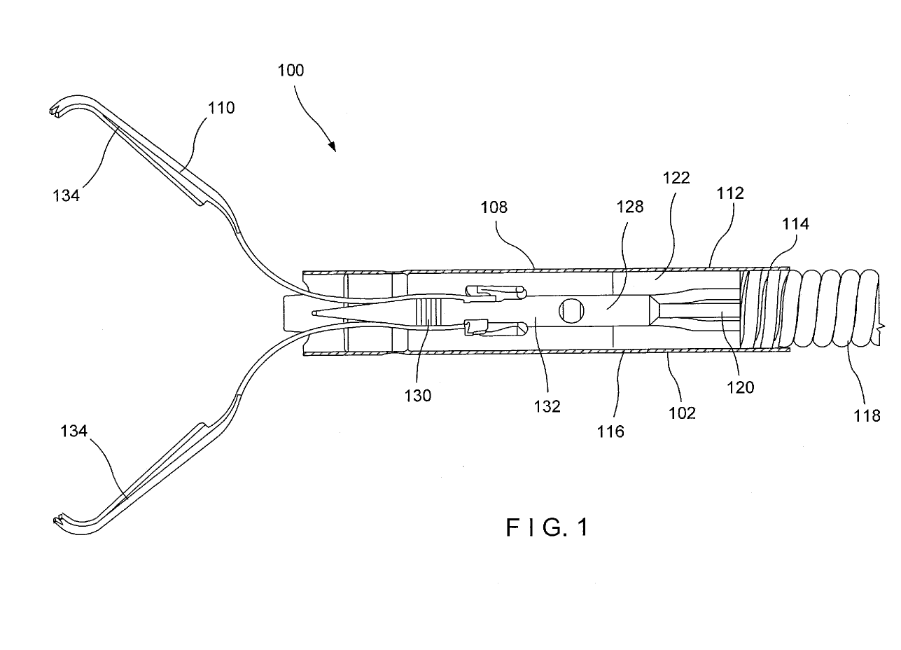

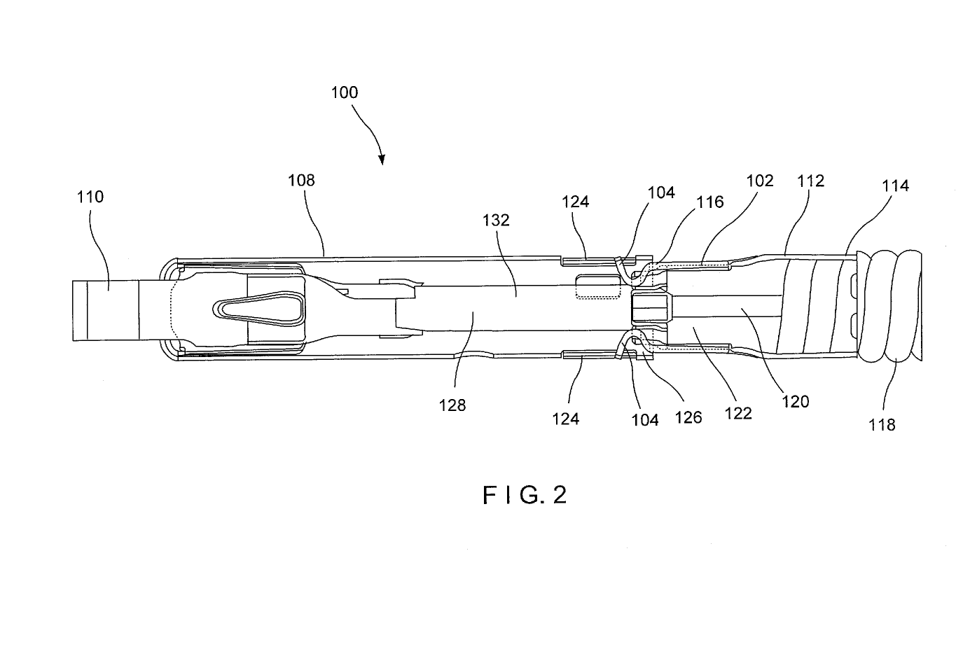

As shown in FIGS. 1-2, a clipping device 100 according to an exemplary embodiment of the present invention comprises a bushing 102 including a plurality of cantilever arms 104 for releasably engaging a capsule 108 and a clip 110 housed therein. The bushing 102 in this embodiment has a substantially cylindrical body 112 extending from a proximal end 114 coupled to a flexible insertion member 118 to a distal end 116 from which the arms 104 extend. However, those skilled in the art will understand that the bushing 102 and capsule 108 may take any shape desired for a particular application without departing from the scope of the invention. The insertion member 118 is an elongated member extending between a proximal end coupled to an actuating member or handle (not shown) and a distal end connected to the bushing 102. As those skilled in the art will understand, the insertion member 118 is formed of a flexible material to allow it to be advanced through a natural body lumen without damaging the tissue thereof and will have a length suited to the requirements of a procedure being performed. The clipping device 100 further comprises a control element 120 extending through the insertion member 118 between the handle and the clip 110. The control element 120 extends through the insertion member 118 and the bushing 102 from a proximal end coupled to the handle to a distal end connected to the clip 110 such that movement of an actuating mechanism of the handle moves the clip 110 between an open and a closed configuration via the handle. For example, as the control member 120 is moved distally relative to the capsule 108, clip arms 134 are extended distally from a distal end of the capsule 108. When extended distally from the capsule 108, the clip aims 134 separate from one another under a natural bias into an open configuration to receive a target tissue therebetween. The control member 120 may be moved proximally relative to the capsule 108 to pull the clip 110 back into the capsule 108 so that contact between the capsule 108 and the clip arms 134 draws the clip arms 134 together into the closed configuration gripping target tissue received therebetween. The control member 120 is coupled to the clip 110 via, for example, a core member 128 including a frangible link 130 designed to fail when subject to a predetermined load. When the link 130 is severed, the clip 110 is locked within the capsule 108 in the closed configuration and the capsule 108 and the clip 110 are separated from the insertion member 118 of the device 100.

Specifically, the bushing 102 includes a channel 122 extending through a substantially cylindrical body 112 with the control member 120 extending therethrough. Cantilever arms 104 extend distally from the distal end 116 to engage windows 124 formed in a proximal end 126 of the capsule 108. In this embodiment, the cantilever arms 104 are hook-shaped to engage the capsule windows 124 and are biased toward a centerline of the bushing 102. The arms 104 are held radially outward in engagement with the windows 124 by a proximal portion 132 of the core member 128 which is received within the distal end of the channel 122 of the bushing 102, as described in U.S. Patent Application Publication No. 2011/0046651. When the core member 128 is received within the bushing 102, the core member 102 forms a plug which pushes the cantilever arms 104 radially outward maintaining them in a locked position binding the bushing 102 to the capsule 108 as the arms 104 engage the capsule windows 124. Once the core member 128 has been severed (i.e., after the frangible link 132 has been broken), the proximal portion is drawn proximally through the capsule 108 and the bushing 102 out of engagement with the cantilever arms 104 permitting the cantilever arms 104 to spring radially inward out of engagement with the windows 124 separating the bushing 102 from the capsule 108. At this point, tabs at a proximal end of the clip 110 spring outward to engage the capsule windows 124 locking the clip 110 in the closed configuration.

The cantilever arms 104 are formed of a material which allows the deformation of the arms 104 as they are urged radially outward into engagement with the windows 124 to be elastic —ensuring that the arms 104 spring radially inward when no longer supported by the core member 128 so that a clip 110 which has been locked over target tissue does not remain coupled to the insertion member 118. For example, the arms 104 may be comprised of 17-7PH stainless in condition RH 950 with a yield stress of 220 ksi. In another example, using another condition such as CH900, the yield stress may be increased to 260 ksi by cold-working the bushing 102. In another exemplary embodiment, the cantilever arms 104 and/or the bushing 102 may comprise Elgiloy or MP35N and may have a yield strength of up to 300 ksi. In order to maximize yield strength, the Elgiloy or MP35N will need to be cold worked and heat treated for precipitation hardening. In an alternate embodiment, the bushing 102 or any portion thereof including the arms 104 may be coated with a material such as Parylene or Teflon to coat the arms 104 and serve as a lubricant to ensure that the cantilever arms 104 disengage the capsule windows 124 as the cantilever arms 104 revert to their biased configuration towards the centerline of the bushing 102.

It will be understood by those of skill in the art that although the exemplary embodiment above specifically describes support of the bushing 102 and release of the capsule 108 and the clip 110 via the core member 132, the core member 132 is not required. The clip 110 may, for example, be directly connected to the control member 120 and may include alternate bushing supports as described in, for example, U.S. Patent Application Publication NO. 2008/0306491. It will also be understood by those of skill in the art that even though the exemplary embodiment specifically describes a clipping device 100, the bushing 102 may be utilized with any medical device in which a proximal portion of the device requires disengagement from a distal portion including a catheter or tube.

It will be apparent to those skilled in the art that various modifications and variations can be made in the structure and methodology of the present invention, without departing from the spirit or scope of the invention. Thus, it is intended that the present invention cover the modifications and variations of this invention provided that they come within the scope of the appended claims and their equivalents.

What is claimed is:

1. A medical device, comprising:
a distal portion sized and shaped for insertion into a body lumen and including a capsule extending longitudinally from a proximal end to a distal end and including a lumen extending therethrough, the proximal end of the capsule including a plurality of windows extending therethrough;
a bushing extending from a proximal end to a distal end and including a channel extending therethrough, the distal end including a plurality of arms extending distally therefrom such that a corresponding one of the arms is releasably engagable with a corresponding one of the windows of the capsule, the arms being biased toward a centerline of the bushing in a release configuration; and
a bushing support receivable within the channel of the bushing to apply a radially outward pressure to the arms such that the arms engage the windows of the capsule in a locked configuration, the arms formed of a material selected to have a yield stress such that movement of the bushing support out of the channel reverts the arms to the release configuration even when the distal portion is inserted through tortuous paths of a body lumen.

2. The device of claim 1, wherein the yield stress of the arms range from between 200 ksi and 300 ksi.

3. The device of claim 1, wherein the arms of the bushing are formed of 17-7 PH stainless in one of condition RH950 and condition CH900.

4. The device of claim 1, wherein the arms of the bushing are formed of Elgiloy or MP35N.

5. The device of claim 1, wherein the arms are treated with a material to reduce friction between the arms and the windows of the capsule.

6. The device of claim 5, wherein the arms are coated with one of Parylene and Teflon.

7. The device of claim 1, wherein a body of the bushing is substantially cylindrical.

8. The device of claim 1, wherein the arms are substantially hook-shaped to engage the windows of the capsule.

9. The device of claim 1, wherein the distal portion includes a clip including a proximal end housed in the capsule such that the clip is movable between a tissue receiving configuration and a tissue gripping configuration via a control member extending through the capsule and the bushing to a proximal end of the device.

10. A tissue clipping device, comprising:
a distal portion sized and shaped for insertion into a body lumen, the distal portion including a clip including a plurality of clip arms, a proximal end of the clip movably housed within a lumen of a capsule extending longitudinally from a proximal end to a distal end such that the clip is movable relative to the capsule between a tissue receiving configuration in which clip arms extend distally from a distal end of the capsule in a biased open position and a tissue gripping configuration in which the arms are retracted proximally within the capsule and restricted by the capsule toward one another, the capsule including a plurality of windows extending laterally through a proximal end thereof;
a bushing extending from a proximal end to a distal end and including a channel extending therethrough, the distal end including a plurality of tabs extending distally therefrom such that a corresponding one of the tabs is releasably engagable with a corresponding one of the windows of the capsule, the tabs being biased toward a centerline of the bushing in a release configuration;
a flexible member extending longitudinally from a distal end attached to the bushing to a proximal end including a handle extending outside of the body to control movement of the clip; and
a bushing support receivable within the channel of the bushing to apply a radially outward pressure to the tabs such that the tabs engage the windows of the capsule in a locked configuration, the arms formed of a material selected to have a yield stress such that movement of the bushing support out of the channel reverts the tabs to the release configuration even when the distal portion is inserted through tortuous paths of a body lumen.

11. The device of claim 10, wherein the yield stress of the tabs range from between 200 ksi and 300 ksi.

12. The device of claim 10, wherein the tabs of the bushing are formed of 17-7 PH stainless in one of condition RH950 and condition CH900.

13. The device of claim 10, wherein the tabs of the bushing are formed of Elgiloy or MP35N.

14. The device of claim 10, wherein the tabs are treated with a material to reduce friction between the arms and the windows of the capsule.

15. The device of claim 14, wherein the tabs are coated with one of Parylene and Teflon.

16. The device of claim 10, wherein a body of the bushing is substantially cylindrical.

17. The device of claim 10, wherein the tabs are substantially hook-shaped to engage the windows of the capsule.

18. The device of claim 10, wherein the bushing support is drawn proximally out of engagement with the tabs of the bushing via a control wire such that the distal portion of the device is disengaged and deployed therefrom in the tissue gripping configuration.
